(19) United States
(12) Patent Application Publication
(10) Pub. No.: US 2025/0275992 A1
(43) Pub. Date: Sep. 4, 2025

(54) TOPICAL FORMULATIONS FOR THERAPEUTICALLY ACTIVE AGENTS

(71) Applicant: OCuSOFT, Inc., Rosenberg, TX (US)

(72) Inventor: Paramita Sarkar, Rosenberg, TX (US)

(21) Appl. No.: 19/069,015

(22) Filed: Mar. 3, 2025

Related U.S. Application Data
(60) Provisional application No. 63/561,052, filed on Mar. 4, 2024.

(51) Int. Cl. A61K 31/69; A61K 9/00; A61K 9/06; A61K 47/10; A61K 47/12; A61K 47/18; A61K 47/32; A61K 47/34

(52) U.S. Cl. CPC A61K 31/69; A61K 9/0014; A61K 9/06; A61K 47/10; A61K 47/12; A61K 47/186; A61K 47/32; A61K 47/34

(57) ABSTRACT

The present invention illustrates formulations designed to deliver active ingredients effectively to the surface of the skin. The formulations comprise therapeutically effective amounts of antihistamine, anti-inflammatory, anesthetic, antioxidant, and anti-eczema active agents. Additional ingredients are included to enhance delivery, stability, and physiological compatibility with the skin. The topical formulations are further configured to be delivered via an applicator to the skin.

### CROSS-REFERENCE TO RELATED APPLICATIONS

This application claims the benefit of Provisional U.S. Patent Application No. 63/561,052, filed Mar. 4, 2024, entitled “Topical Formulations for Therapeutically Active Agents”, the entire content and disclosure of which, both express and implied, is incorporated herein by reference.

### TECHNICAL FIELD

The invention relates generally to the field of topically applied formulations and, in particular, to formulations comprising therapeutically active agents.

### BACKGROUND

Topical formulations are designed to effectively deliver active ingredients to the target site on the skin, while providing maximum therapeutic benefits with minimal irritation or adverse effects. The formulation should possess an optimal balance between occlusivity and breathability, ensuring sufficient moisture retention to aid in the healing process without causing excessive occlusion that could exacerbate certain skin conditions.

Ideally, the formulation should have a pleasant texture, easy spreadability, and quick absorption to encourage patient adherence. It should also be hypoallergenic, and free from common irritants and allergens, such as fragrances, to minimize the risk of sensitization. Formulations should also be stable over a wide range of environmental conditions, including temperature and humidity, and possess a suitable shelf-life. Ultimately, the ideal topical dermatological formulation should provide a safe, effective, and patient-friendly means of treating various skin conditions, while maximizing patient satisfaction and overall treatment outcomes.

### SUMMARY

According to an embodiment, the present invention involves formulations comprising therapeutically effective amounts of antihistamine, anti-inflammatory, anesthetic, antioxidant, and anti-eczema agents to the surface of the skin.

According to one or more embodiments, a topical composition for treating eczema includes a PDE4 inhibitor in a stable and effective formulation. The composition comprises a PDE4 inhibitor, a solubilizer, a humectant, preservatives, buffering agents, and purified water.

In one embodiment, the PDE4 inhibitor used is crisaborole at a concentration of 0.1-2.0% w/w. The solubilizer is selected from the group consisting of isopropanol and ethanol and is present in a concentration range of 1.0-25.0% w/w. Humectants are chosen from the group consisting of propylene glycol and glycerin and are at a concentration of 0.5-10.0% w/w.

To maintain the stability of the composition, preservatives such as lauramine oxide, 1,2-hexanediol, and 1,2-octanediol are present in the composition, with each in the range of 0.01-2.0% w/w. Buffering agents comprise sodium citrate, citric acid, sodium acetate, acetic acid, sodium phosphate monobasic, and sodium phosphate dibasic at concentrations ranging from 0.05-5.0% w/w, and the overall pH of the composition is maintained within a range of 4.5-7.5.

The composition can be applied to the skin for the treatment of eczema. In some embodiments, the composition is applied using an applicator such as a wipe, cloth, pad, sponge, brush, swab, puff, or roll-on applicator.

According to an exemplary embodiment, a formulation for treating eczema includes 0.1-2.0% w/w crisaborole, 1.0-25.0% w/w ethanol, 0.5-10.0% w/w propylene glycol, 0.1-3.0% w/w carbomer, 0.01-1.0% w/w lauramine oxide, 0.05-5.0% w/w sodium citrate, 0.05-1.0% w/w citric acid, and purified water. This composition is designed to be stable, effective, and easy to apply for managing eczema.

### DETAILED DESCRIPTION

The term and phrases “invention,” “present invention,” “instant invention,” and similar terms and phrases as used herein are non-limiting and are not intended to limit the present subject matter to any single embodiment, but rather encompass all possible embodiments as described.

As used herein, all weight percentages (wt. %) are based on the total wt. % of the skin care composition, unless otherwise specified. Additionally, all composition percentages are based on totals equal to 100 wt. %, unless otherwise specified.

The systems and methods for their use can “comprise,” “consist essentially of,” or “consist of” any of the ingredients or steps disclosed throughout the specification. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and can include the ingredients of the present invention and do not exclude other ingredients or elements described herein. The use of the word “a” or “an” when used in conjunction with the term “comprising” in the claims and/or the specification may mean “one,” but it is also consistent with the meaning of “one or more,” “at least one,” and “one or more than one.” As used herein, “consisting essentially of” means that the invention may include ingredients in addition to those recited in the claim, but only if the additional ingredients do not materially alter the basic and novel characteristics of the claimed invention. Generally, such additives may not be present at all or only in trace amounts. However, it may be possible to include up to about 10% by weight of materials that could materially alter the basic and novel characteristics of the invention as long as the utility of the composition (as opposed to the degree of utility) is maintained.

As used herein, the term “therapeutically effective amount” of a composition or active ingredient refers to an amount sufficient to elicit the desired biological response. The terms composition and active ingredient are used interchangeably herein. As will be appreciated by those of ordinary skill in this art, and effective amount of a substance may vary depending on such factors as the desired biological endpoint, the patient, etc. The terms effective amount and therapeutically effective amount may be used interchangeably herein.

According to one or more embodiments, the present invention involves a composition containing a therapeutically effective amount of one or more active agents and is configured for application to the surface of the skin. The active agent can include, without limitation, antihistamine, anti-inflammatory, anesthetic, antioxidant, and anti-eczema active agents or mixtures thereof.

Antihistamines are a class of active agents used to relieve the symptoms of allergic skin conditions, such as hives and allergic dermatitis. The agents work by blocking the action of histamine, a chemical released by the body in response to an allergen that causes itching, swelling, and redness. According to an embodiment, a topical antihistamine formulation includes one or more agents chosen from the group comprising chlorpheniramine, azelastine, diphenhydramine, and loratadine. The concentration of each of the antihistamine agents is in the range of about 0.005 to about 5% w/w.

Anti-inflammatory agents are employed to reduce inflammation and irritation in the skin. These agents work by suppressing the immune system and decreasing the production of inflammatory chemicals in the skin. They are often used to treat conditions such as psoriasis and dermatitis. According to an embodiment, a topical anti-inflammatory composition includes one or more agents chosen, without limitation, from the group consisting of methyl salicylate, ketorolac, diclofenac, and loteprednol etabonate. The concentration of each of the anti-inflammatory agents is in the range of about 0.025 to about 15% w/w.

Anesthetics are agents that numb the skin to alleviate pain during routine procedures, such as injections, biopsies, and minor surgeries. When an anesthetic is applied to the skin or subcutaneous tissue, it diffuses into the nerve endings and, subsequently, prevents the nerve from transmitting pain signals to the brain. According to an embodiment, an anesthetic composition includes one or more agents chosen from the group comprising tetracaine, proparacaine, and benoxinate. The concentration of each of the anesthetic agents is in the range of about 0.25 to about 3% w/w.

Anti-eczema agents can be applied to the skin to reduce inflammation and the itching associated with eczema. These agents work by decreasing production of inflammatory chemicals in the skin and by blocking the immune response that leads to eczema symptoms. According to an embodiment, an anti-eczema composition includes one or more agents chosen from the group comprising crisaborole, pramoxine, calamine, hydrocortisone, clobetasol propionate. The concentration of each of the anti-eczema agents is in the range of about 0.025 to about 10% w/w.

Phosphodiesterase enzymes catalyze the hydrolysis of phosphodiester bonds in cyclic nucleotides, DNA, RNA, and other small molecules containing the target functional group. The enzymatic activity of phosphodiesterases is critical for the regulation of signal transduction and, due to their localized distribution in human tissue and known active site structures, are often targeted to mitigate the pathogenesis of many human diseases. In particular, the phosphodiesterase-4 (PDE4) family of enzymes regulate the level of cyclic adenosine monophosphate (cAMP) in tissues thereby modulating inflammatory responses. Inflammatory diseases such as eczema benefit from PDE4 inhibition, which reduces inflammation and relieves symptoms, including itching, redness, and swelling.

Crisaborole is a nonsteroidal, low molecular weight phenoxybenoxaborole compound that effectively penetrates the skin at the site of application. It inhibits the PDE4 family of enzymes subsequently reducing degradation of cAMP. As an anti-eczema agent, crisaborole advantageously avoids common side-effects associated with corticosteroid therapy, such as skin thinning, worsened acne, and atrophy. These adverse effects become more pronounced with long-term steroid use, limiting tolerability and safety. Conversely, crisaborole is safe for long-term use as an anti-eczema agent and has negligible off-target activity, as it is quickly hydrolyzed into inactive metabolites. In an exemplary embodiment, crisaborole is present in the range of 0.1-2.0% w/w.

In one or more embodiments, the topical formulation comprises additional ingredients to enhance the delivery and stability of the active agent and to also enhance the physiological compatibility with the skin. These ingredients comprise, without limitation, surfactants, antioxidants, emollients, buffering agents, preservatives, viscosity enhancers, humectants, solubilizers, and an aqueous vehicle.

An organic solvent may be added to the composition as a solubilizer to help dissolve components that are insoluble in the selected vehicle. Suitable organic solvents are alcohols with low molecular weights, such as isopropanol and ethanol. The low molecular weight alcohols can help the composition feel lighter on the skin, set quickly, and give a cooling effect. In one or more embodiments, the solubilizer is ethanol in the range of 1.0-25.0% w/w.

Humectants are moisturizing agents that promote the retention of water due to their hygroscopic character. Compounds such as propylene glycol and glycerin help heal and prevent skin dryness and irritation by attracting and maintaining moisture. In one or more embodiments, the concentration of propylene glycol is in the range of 0.5-10.0% w/w.

Viscosity enhancers or agents may be added to the composition to achieve a preferred viscosity. In an exemplary embodiment, carbomer is used as a viscosity enhancer in the range of 0.1-3.0% w/w.

The composition can further comprise a preservative having effective anti-microbial activity. Preservatives with significant anti-microbial properties are lauramine oxide and 1,2 diols. In one or more embodiments, the preservative is lauramine oxide in the range of 0.01-1.0% w/w. Preferred 1,2-diols are selected from the group consisting of 1,2-hexanediol and 1,2-octanediol.

The composition may also include buffering agents to maintain a stable pH. Suitable buffering agents include citric acid and sodium citrate, which help regulate pH and enhance composition stability. In one or more embodiments, the buffering agents are present in a concentration range of 0.05-5.0% w/w and the pH is in the range of 4.5-7.5.

Table 1 illustrates an exemplary embodiment for an anti-eczema formulation.

TABLE 1

| Ingredient | Purpose | % w/w |
|---|---|---|
| Crisaborole | Anti-eczema | 0.1-2.0% |
| Ethanol | Solubilizer | 1.0-25.0% |
| Propylene glycol | Humectant | 0.5-10.0% |
| Carbomer | Viscosity enhancer | 0.1-3.0% |
| Lauramine oxide | Preservative | 0.01-1.0% |
| Sodium citrate | Buffering agent | 0.05-5.0% |
| Citric acid | Buffering agent | 0.05-1.0% |
| Purified water | Vehicle | q.s. to 100% |

Table 2 illustrates an exemplary embodiment for an antihistamine formulation.

TABLE 2

| Ingredient | Purpose | % w/w |
|---|---|---|
| Chlorpheniramine | Antihistamine | 0.005-5.0% |
| Polysorbate 80 | Surfactant | 0.05-2.0% |
| Glycerin | Humectant | 0.5-10% |
| Sodium acetate | Buffering agent | 0.05-5.0% |
| Acetic acid | Buffering agent | 0.05-1.0% |
| Purified water | Vehicle | q.s. to 100% |

Table 3 describes an exemplary embodiment for an anti-inflammatory formulation.

TABLE 3

| Ingredient | Purpose | % w/w |
|---|---|---|
| Methyl salicylate | Anti-inflammatory | 1.0-15.0% |
| Polysorbate 80 | Surfactant | 0.05-2.0% |
| Glycerin | Humectant | 0.5-10% |
| Hexylene glycol and | Preservative | 0.4-2.0% |
| caprylyl glycol | | |
| Sodium phosphate monobasic | Buffering agent | 0.05-5.0% |
| Sodium phosphate dibasic | Buffering agent | 0.05-1.0% |
| Purified water | Vehicle | q.s. to 100% |

Table 4 demonstrates an exemplary embodiment for an anesthetic formulation.

TABLE 4

| Ingredient | Purpose | % w/w |
|---|---|---|
| Tetracaine | Anesthetic | 0.1-2.0% |
| Tyloxapol | Surfactant | 0.05-2.0% |
| Glycerin | Humectant | 0.5-10% |
| Hydroxypropylmethyl | Viscosity enhancer | 0.3-3.0% |
| cellulose | | |
| Lauramine oxide | Preservative | 0.01-1.0% |
| Sodium phosphate monobasic | Buffering agent | 0.05-5.0% |
| Sodium phosphate dibasic | Buffering agent | 0.05-1.0% |
| Purified water | Vehicle | q.s. to 100% |

According one or more embodiments, an applicator is used to deliver a therapeutically effective amount of a topical formulation to the skin. The applicator can take different forms, including wipes, cloths, pads, sponges, brushes, swabs, puffs, roll-on applicators, and other similar devices. The invention encompasses a range of materials and fabrics that can be used for the applicator, including natural or synthetic fibers and non-woven fabrics made from different materials.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only.

1. A composition for treating eczema comprising:
a PDE4 inhibitor;
a solubilizer;
a humectant;
one or more preservatives;
one or more buffering agents; and
purified water.

2. The composition for treating eczema according to claim 1, wherein the PDE4 inhibitor is crisaborole.

3. The composition for treating eczema according to claim 2, wherein the concentration of crisaborole is 0.1-2.0% w/w.

4. The composition for treating eczema according to claim 1, wherein the solubilizer is chosen from the group consisting of isopropanol and ethanol.

5. The composition for treating eczema according to claim 1, wherein the concentration of the solubilizer is in the range of 1.0-25.0% w/w.

6. The composition for treating eczema according to claim 1, wherein the humectant is chosen from the group consisting of propylene glycol and glycerin.

7. The composition for treating eczema according to claim 1, wherein the concentration of the humectant is in the range of 0.5-10.0% w/w.

8. The composition for treating eczema according to claim 1, wherein the one or more preservatives is chosen from the group consisting of lauramine oxide, 1,2-hexanediol, and 1,2-octanediol.

9. The composition for treating eczema according to claim 1, where in the concentration of each of the one or more preservatives is in the range of 0.01-2.0% w/w.

10. The composition for treating eczema according to claim 1, wherein the one or more buffering agents is chosen from the group consisting of sodium citrate, citric acid, sodium acetate, acetic acid, sodium phosphate monobasic, and sodium phosphate dibasic.

11. The composition for treating eczema according to claim 1, wherein the concentration of each of the one or more buffering agents is in the range of 0.05-5.0% w/w.

12. The composition for treating eczema according to claim 1, wherein the pH of the composition is in the range of 4.5-7.5.

13. A method for managing eczema, the method comprising the application of an effective amount of the composition according to claim 1 to the skin using an applicator.

14. A method for managing eczema according to claim 13, wherein the applicator is a wipe, cloth, pad, sponge, brush, swab, puff, or roll-on applicator.

15. A composition for treating eczema comprising:
0.1-2.0% w/w crisborole;
1.0-25.0% w/w ethanol;
0.5-10.0% w/w propylene glycol;
0.1-3.0% w/w carbomer;
0.01-1.0% w/w lauramine oxide;
0.05-5.0% w/w sodium citrate;
0.05-1.0% w/w citric acid; and
purified water q.s. to 100%.
